(19) United States
(12) Patent Application Publication
(10) Pub. No.: US 2025/0288585 A1
(43) Pub. Date: Sep. 18, 2025

(54) SYNERGISTIC COMPOSITIONS FOR ENHANCED HAIR GROWTH AND METHODS FOR TREATING HAIR LOSS

(71) Applicant: The Johns Hopkins University, Baltimore, MD (US)

(72) Inventor: Crystal AGUH, Baltimore, MD (US)

(21) Appl. No.: 19/080,324

(22) Filed: Mar. 14, 2025

Related U.S. Application Data
(60) Provisional application No. 63/565,845, filed on Mar. 15, 2024.

(51) Int. Cl. A61K 31/506; A61K 9/06; A61K 31/07; A61K 31/203; A61K 31/573; A61P 17/14

(52) U.S. Cl. CPC A61K 31/506; A61K 9/06; A61K 31/07; A61K 31/203; A61K 31/573; A61P 17/14

(57) ABSTRACT

The presently disclosed subject matter generally relates to synergistic compositions for enhanced hair growth and methods for treating hair loss.

### FIELD

The present application claims priority to U.S. Provisional Application No. 63/565,845, filed Mar. 15, 2024, which is incorporated herein by reference in its entirety.

### FIELD

The present disclosure relates to compositions for enhanced hair growth and methods for treating hair loss.

### BACKGROUND

Alopecia is a disease in which hair is lost. There are various types of alopecia, including, but not limited to: alopecia areata, androgenetic alopecia, postmenopausal alopecia, female pattern alopecia, seborrheic alopecia, alopecia pityroides, senile alopecia, cancer chemotherapy drug-induced alopecia, alopecia due to radiation exposure, trichotillomania, postpartum alopecia, traction alopecia, and the like. These types of alopecia have the same symptoms of hair loss, but are based on different causes, and therapies therefore can be different from each other.

Black women suffer from alopecia more than any other group. More than two-thirds of black women suffer from hair loss. Traction alopecia is one of the most common forms of hair loss in black women, affecting up to one-third of black women. This condition occurs as a result of use of high tension hair styles.

Topical minoxidil is FDA-approved for androgenetic alopecia in men and women (i.e., male pattern baldness and female pattern baldness), and approved for over-the-counter use as a foam (5%) or solution (5% and 2%) formulation. Long-term use of minoxidil is required to sustain results.

Use of over-the-counter minoxidil solution, however, results in breakage in curly hair. Further, over-the-counter minoxidil foam leads to build up in curly hair. Thus, patient compliance with regular and long-term use of minoxidil to treat alopecia in subjects with curly hair is problematic.

### SUMMARY

The presently disclosed subject matter generally relates to synergistic compositions for enhanced hair growth and methods for treating hair loss. The compositions disclosed herein comprise a synergistic combination of minoxidil, a retinoic acid, and a steroid. It has surprisingly been found that the compositions disclosed herein have a synergistic effect of enhanced hair growth when applied in combination; that is, the components are more effective in combination than when applied individually.

In some aspects, the composition is formulated as an oil or ointment for topical administration. The compositions disclosed herein are particularly effective for treating alopecia in subjects with curly hair due to increased patient compliance with regular and long-term use of the composition as compared to current over-the-counter foam (5%) or solution (5% and 2%) formulations.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 are photos of an individual with traction alopecia and type 4 curly/kinky hair.

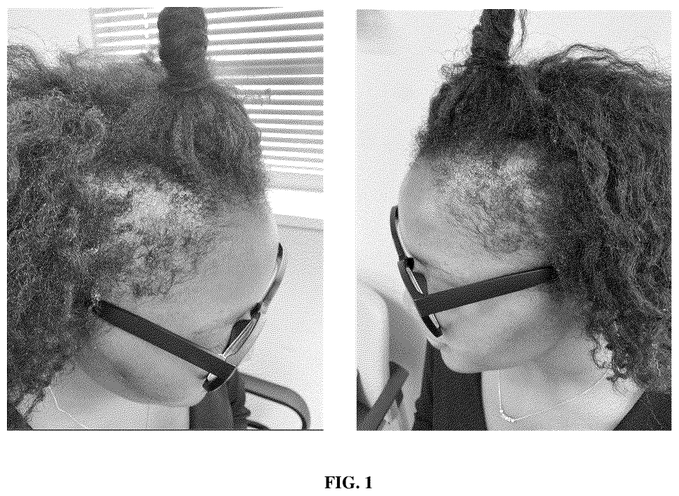

FIG. 2 are photos of a patient's central scalp (Patient 1) before treatment (left) and after treatment (right) with a composition comprising 7% minoxidil, 0.025% tretinoin and fluocinolone 0.01% oil. The composition was an oil. The patient applied a few drops to affected scalp areas once daily. The patient has type 4 curly/kinky hair.

FIG. 3 are photos of a patient's scalp (Patient 2) before treatment (left) and after treatment (right) with a composition comprising 8% minoxidil, 0.025% tretinoin and triamcinolone 0.1% ointment for 4 months. The composition was an ointment. The patient applied a pea sized amount to affected scalp areas once daily. The patient has type 4 curly/kinky hair. The compounded minoxidil in an ointment base enhances compliance over 4 months, as based on patient reporting.

FIG. 4 are photos of a patient's scalp (Patient 3) before treatment (left) and after treatment (right) with a composition comprising 10% minoxidil, 0.025% tretinoin and triamcinolone 0.1% ointment for 4 months. The composition was an ointment. The patient applied a pea sized amount to the affected scalp areas once daily. The patient has type 4 curly/kinky hair.

FIG. 5 are photos of a patient's scalp (Patient 4) before treatment (left) and after treatment (right) with a composition comprising 10% minoxidil, 0.025% tretinoin and triamcinolone 0.1% ointment. The composition was an ointment. The patient applied a pea sized amount to the affected scalp areas once daily. The patient has type 4 curly/kinky hair. The patient shown in FIG. 5 had previously tried over the counter minoxidil consistently and showed no improvement and presented with severe hair loss prior to treatment with the composition comprising 10% minoxidil, 0.025% tretinoin and triamcinolone 0.1% ointment.

FIG. 6 are photos of a patient's scalp (Patient 5) before treatment (left) and after treatment (right) with a composition comprising 10% minoxidil, 0.025% tretinoin and triamcinolone 0.1% ointment for 1 year. The composition was an ointment. The patient applied a pea sized amount to affected scalp areas once daily. The patient has type 4 curly/kinky hair.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Therefore, it is to be understood that the presently disclosed subject matter is not to be limited to the specific embodiments disclosed and that modifications and other embodiments are intended to be included within the scope of the appended claims.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, and the like). Furthermore, all values within a given range may be an endpoint for the range encompassed thereby (e.g., the range 50-80 includes the ranges with endpoints such as 55-80, 50-75, and the like). The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The term “about” as used herein includes a 1, %, 2%, 3%, 4%, 5%, 10%, 15%, or 20% deviation above or below the given value. For an amount of an active ingredient in a composition provided herein, the term “about” refers to a 5%, 10%, 20% or 30% deviation above or below the given value. For example, an amount of an active ingredient in a composition given as “about 1.0% w/w” includes a range of 0.95% to 1.05% w/w, 0.9% to 1.1% w/w, 0.8% to 1.2% w/w, or 0.7% to 1.3% w/w. For an amount of an inactive ingredient or excipient in a pharmaceutical formulation provided herein, the term “about” refers to a 1, %, 2%, 3%, 4%, 5%, 10%, 15%, or 20% deviation above or below the given value. One skilled in the art is able to determine reasonable deviations based on the specific value evaluated. The range encompassed by the term “about” is informed by the context of the specification and claims, which both describe the range of specific activity covered by the term “about,” i.e. treating hair loss.

The “% w/w” refers to percentage by weight of active components relative to the total weight of the composition.

As used herein, the term “consists of” or “consisting of” means that the formulation includes only the elements, steps, or ingredients specifically recited in the particular claimed embodiment or claim.

As used herein, the term “consisting essentially of” or “consists essentially of” means that the only active pharmaceutical ingredient in the composition or method that treats the specified condition (e.g. hair loss) is the specifically recited therapeutic in the particular embodiment or claim. A synergistic composition consisting essentially of minoxidil in an amount ranging from about 7% to about 10% w/w, including about 7%, 7.5%, 8%, 8.5%, 9%, 0.5%, and 10% w/w minoxidil, a retinoid agent in an amount ranging from 0.025% to 0.1% w/w, including 0.025%, 0.05%, 0.75%, and 0.1% w/w retinoid agent, and a pharmaceutically acceptable carrier is limited to the recited components and other component(s) or ingredient(s) that do not materially affect the basic and novel characteristics of the synergistic composition. The basic and novel characteristics include the synergistic effect when minoxidil and a retinoid agent are applied in combination; the components are more effective in combination than when applied individually. A synergistic composition consisting essentially of minoxidil in an amount ranging from about 7% to about 10% w/w, including about 7%, 7.5%, 8%, 8.5%, 9%, 0.5%, and 10% w/w minoxidil, a retinoid agent in an amount ranging from 0.025% to 0.1% w/w, including 0.025%, 0.05%, 0.75%, and 0.1% w/w retinoid agent, a steroid in an amount ranging from about 0.01% to about 0.1% w/w, including about 0.01%, 0.025%, 0.05%, 0.075%, and 0.1% w/w steroid, and a pharmaceutically acceptable carrier is limited to the recited components and other component(s) or ingredient(s) that do not materially affect the basic and novel characteristics of the synergistic composition. The basic and novel characteristics include the synergistic effect when minoxidil, a retinoid agent, and a steroid are applied in combination; the components are more effective in combination than when applied individually.

More particularly, the term “combination” is used to refer to the active agents combined and administered in a single dosage form.

Further, a composition disclosed herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a composition disclosed herein and at least one additional therapeutic agent can be varied so long as the beneficial effects of the combination of these agents are achieved. Accordingly, the phrase “in combination with” refers to the administration of a composition described herein and at least one additional therapeutic agent either simultaneously, sequentially, or a combination thereof. Therefore, a subject administered a combination of a composition described herein and at least one additional therapeutic agent can receive the composition and at least one additional therapeutic agent at the same time (i.e., simultaneously) or at different times (i.e., sequentially, in either order, on the same day or on different days), so long as the effect of the combination of both agents is achieved in the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the composition described herein and at least one additional therapeutic agent are administered simultaneously, they can be administered to the subject as separate pharmaceutical compositions, or they can be administered to a subject as a single pharmaceutical composition comprising both agents. The terms “treating” and “treatment” as used herein refers to reduction in severity and/or frequency of symptoms, elimination of symptoms and/or underlying cause, and improvement or remediation of damage caused thereby. Accordingly, the term “treating” can include reversing, alleviating, inhibiting the progression of, preventing, or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The presently disclosed subject matter provides a synergistic composition for enhancing hair growth comprising minoxidil in an amount ranging from about 7% to about 10% w/w, including about 7%, 7.5%, 8%, 8.5%, 9%, 0.5%, and 10% w/w minoxidil, and a retinoid agent in an amount ranging from about 0.025% to about 0.1% w/w, including 0.025%, 0.05%, 0.75%, and 0.1% w/w retinoid agent. In some embodiments, the composition further comprises a steroid in an amount ranging from about 0.01% to about 0.1% w/w, including about 0.01%, 0.025%, 0.05%, 0.075%, and 0.1% w/w steroid.

The presently disclosed subject matter provides a synergistic composition for enhancing hair growth consisting of minoxidil in an amount ranging from about 7% to about 10% w/w, including about 7%, 7.5%, 8%, 8.5%, 9%, 0.5%, and 10% w/w minoxidil, and a retinoid agent in an amount ranging from about 0.025% to about 0.1% w/w, including 0.025%, 0.05%, 0.75%, and 0.1% w/w retinoid agent. The presently disclosed subject matter provides a synergistic composition for enhancing hair growth consisting of minoxidil in an amount ranging from about 7% to about 10% w/w, including about 7%, 7.5%, 8%, 8.5%, 9%, 0.5%, and 10% w/w minoxidil, a retinoid agent in an amount ranging from about 0.025% to about 0.1% w/w, including 0.025%, 0.05%, 0.75%, and 0.1% w/w retinoid agent, and a steroid in an amount ranging from about 0.01% to about 0.1% w/w, including about 0.01%, 0.025%, 0.05%, 0.075%, and 0.1% w/w steroid. The presently disclosed subject matter provides a synergistic composition for enhancing hair growth consisting of minoxidil, a retinoid agent, and a steroid.

The presently disclosed subject matter provides a synergistic composition for enhancing hair growth consisting essentially of minoxidil in an amount ranging from about 7% to about 10% w/w, including about 7%, 7.5%, 8%, 8.5%, 9%, 0.5%, and 10% w/w minoxidil, and a retinoid agent in an amount ranging from about 0.025% to about 0.1% w/w, including 0.025%, 0.05%, 0.75%, and 0.1% w/w retinoid agent. The presently disclosed subject matter provides a synergistic composition for enhancing hair growth consisting essentially of minoxidil in an amount ranging from about 7% to about 10% w/w, including about 7%, 7.5%, 8%, 8.5%, 9%, 0.5%, and 10% w/w minoxidil, a retinoid agent in an amount ranging from about 0.025% to about 0.1% w/w, including 0.025%, 0.05%, 0.75%, and 0.1% w/w retinoid agent, and a steroid in an amount ranging from about 0.01% to about 0.1% w/w, including about 0.01%, 0.025%, 0.05%, 0.075%, and 0.1% w/w steroid. The presently disclosed subject matter provides a synergistic composition for enhancing hair growth consisting essentially of minoxidil, a retinoid agent, and a steroid.

### Minoxidil

Minoxidil is considered a first line therapy for various forms of non-scarring alopecia and can increase hair growth on the scalp. Topical minoxidil is FDA-approved for androgenetic alopecia in men and women (i.e., male pattern baldness and female pattern baldness), and approved for over-the-counter use as a foam (5%) or solution (5% and 2%) formulation. The effectiveness of minoxidil, however, is widely variable.

“Minoxidil acts through multiple pathways (vasodilator, anti-inflammatory agent, inducer of the Wnt/b-catenin signaling pathway, an antiandrogen), and may also affect the length of the anagen and telogen phases. Approximately 1.4% of topical minoxidil is absorbed through the skin. Minoxidil is a prodrug that is metabolized by follicular sulfotransferase to minoxidil sulfate (active form). Those with higher sulfotransferase activity may respond better than patients with lower sulfotransferase activity.” A.K. Gupta, M. Talukder, M. Venkataraman & M. A. Bamimore (2022) Minoxidil: a comprehensive review, Journal of Dermatological Treatment, 33:4, 1896-1906. Gupta et al. also discloses that efficacy of the minoxidil solution may not correlate with increased concentration.

A randomized control trial found 10% minoxidil solution less effective than 5%, and 10% minoxidil solution caused more scalp irritation and shedding due to the high percentage of PEG used in the formulation. Ghonemy S, Alarawi A, Bessar H. Efficacy and safety of a new 10% topical minoxidil versus 5% topical minoxidil and placebo in the treatment of male androgenetic alopecia: a trichoscopic evaluation. J Dermatolog Treat. 2021; 32 (2): 236-241.

Accordingly, it is surprising that the synergistic compositions disclosed herein comprising higher concentrations of minoxidil (in combination with a retinoid agent, and in some embodiments, a steroid) demonstrate enhanced hair growth.

In some embodiments, the synergistic composition comprises minoxidil in an amount of about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, or about 11% w/w. In some embodiments, the composition comprises minoxidil in an amount ranging from about 7% to about 10% w/w.

### Retinoid Agent

In some embodiments, the retinoid agent comprises a retinyl ester, retinol, retinaldehyde, or a retinoic acid. The retinoid acid can include all-trans-retinoic acid, 9-cis-retinoic acid, and 13-cis-retinoic acid. In some embodiments, the retinoid agent comprises a retinoic acid selected from tazarotene, isotretinoin, adapalene, tretinoin, trifarotene, alitretinoin, and bexarotene.

In some embodiments, the composition comprises a retinoid agent in an amount of 0.025% to 0.1% w/w. In some embodiments, the composition comprises a retinoid agent in an amount of 0.01% w/w, 0.015% w/w, 0.02% w/w, 0.025% w/w, 0.03% w/w, 0.035% w/w, 0.04% w/w, 0.045% w/w, 0.05% w/w, 0.055% w/w, 0.06% w/w, 0.065% w/w, 0.07% w/w, 0.075% w/w, 0.08% w/w, 0.085% w/w, 0.09% w/w, 0.095% w/w, or 0.10% w/w.

Retinyl esters, retinol and retinaldehyde are the three precursors to retinoic acid. The conversion sequence is a retinyl ester to retinol, which gets converted to retinaldehyde, giving rise to the final product, retinoic acid. The biologically active form, retinoic acid, is what leads to the improvement in skin texture, fine lines and dyspigmentation.

Treinoin is indicated in the treatment of acne, acne vulgaris, and photoaging/rhytides. It is also used off-label to treat keratosis pilaris, actinic keratosis, and hyperpigmentation (melasma, solar lentigines). Tazarotene is indicated in acne vulgaris and plaque psoriasis. Adapalene is a topical retinoid indicated in acne vulgaris and off-label it is also used in the treatment of hyperpigmentation and actinic keratosis, and due to its tolerability, it is often used off-label for photoaging/rhytides. Trifarotene is indicated for acne vulgaris of the face and trunk. Motamedi M, Chehade A, Sanghera R, Grewal P. A Clinician's Guide to Topical Retinoids. Journal of Cutaneous Medicine and Surgery. 2022;26 (1): 71-78.

In 1986, Bazzano et al. tested topical all-trans-retinoic acid (tretinoin) alone and in combination with 0.5% minoxidil for the promotion of hair growth in subjects with androgenetic alopecia. Bazzano et al reported that a) “[a] fter 1 year, the combination of topical tretinoin with 0.5% minoxidil resulted in terminal hair regrowth in 66% of the subjects studied;” and b) “[t] retinoin was shown to stimulate some hair regrowth in approximately 58% of the subjects studied.” Terminal hair is thick, coarse hair that grows on the face, armpits, pubic area, chest, and abdomen. Bazzano GS, Terezakis N, Galen W. Topical tretinoin for hair growth promotion. J Am Acad Dermatol. 1986; 15 (4 Pt 2): 880-893.

In 1990, Ferry et al. reported results from a three-way, randomized, crossover study to determine the effect of the synthetic retinoid, tretinoin, on percutaneous absorption of minoxidil. “[Nineteen healthy male subjects] received, for 20 days, twice-daily administrations of 1 mL of an aqueous 2% topical minoxidil solution either alone, with once-daily applications of a 0.05% tretinoin cream, or with once-daily applications of a vehicle control cream. When minoxidil was co-administered with tretinoin cream, minoxidil absorption was increased nearly threefold, compared with a 1.3-fold increase in absorption observed with coadministration of vehicle control cream.” Ferry JJ, Forbes KK, VanderLugt JT, Szpunar GJ. Influence of tretinoin on the percutaneous absorption of minoxidil from an aqueous topical solution. Clin Pharmacol Ther. 1990 April;47 (4): 439-46.

In September 2007, however, Shin et al. reported results of a randomized, double-blind, comparative clinical trial that “compare [d] the efficacy and safety of therapy using a combined solution of 5% minoxidil and 0.01% tretinoin once daily with those of the conventional 5% topical minoxidil therapy applied twice daily in the treatment of AGA.” Shin et al. reported that “[t] here were no statistically significant differences between the two treatment groups with respect to changes in macrophotographic variables or scores on subjective global assessments by patients and the investigator. The incidence of adverse effects such as pruritus or local irritation was similar in the 5% minoxidil group (4 of 14 subjects) and the combined agent group (5 of 15 subjects).” Shin et al. Efficacy of 5% minoxidil versus combined 5% minoxidil and 0.01% tretinoin for male pattern hair loss: a randomized, double-blind, comparative clinical trial. Shin HS, Won CH, Lee SH, Kwon OS, Kim KH, Eun HC., Am J Clin Dermatol. 2007; 8 (5): 285-90. Shin et al.'s robust clinical study demonstrated no improvement of a combined solution of 5% minoxidil and 0.01% tretinoin once daily over conventional 5% topical minoxidil therapy.

Other in vitro studies suggest use of tretinoin in combination with minoxidil:
Sharma A, Goren A, Dhurat R, et al. Tretinoin enhances minoxidil response in androgenetic alopecia patients by upregulating follicular sulfotransferase enzymes. Dermatol Ther. 2019; 32 (3): e12915 (studied the effect of topical tretinoin on follicular sulfotransferase) in vitro via the Minoxidil Response Test utilizing plucked hairs (Follea Ltd., Munich, Germany), an in vitro medical device for determining a patient's response to topical minoxidil wherein the value from the test indicates the level of sulfotransferase activity present in the sampled hairs; and reported “topical [0.1%] tretinoin application influences the expression of follicular sulfotransferase” in vitro).
Kwon, O., S., Pyo, H., K., Oh, Y., J., Han, J., H., Lee, S., R., Chung, J., H., Eun, H., C., Kim, K., H. (April 2007). Promotive effect of minoxidil combined with all-trans retinoic acid (tretinoin) on human hair growth in vitro. Journal of Korean Medical Science, 22, 283-289.
Yoo, H., G., Chang, I., Y., Pyo, H., K., Kang, Y., J., Lee, S., H., Kwon, O., S., Cho, K., H., Eun, H., C., Kim, K., H. (January 2007). The additive effects of minoxidil and retinol on human hair growth in vitro. Biological and Pharmaceutical Bulletin, 30, 21-26.
Voorhees, J., J. (1990). Clinical effects of long-term therapy with topical tretinoin and cellular mode of action. Journal of International Medical Research, J Int Med Res. 1990;18 Suppl 3: 26C-28C.
Maiti, S., Chen, X., Chen, G. (2005). All-trans retinoic acid induction of sulfotransferases. Basic & Clinical Pharmacology & Toxicology, 96, 44-53.

These in vitro studies, however, ultimately do not discount the Shin et al. in vivo clinical results that show that there is no statistically significant difference between a treatment using a combined solution of 5% minoxidil and 0.01% tretinoin once daily and the conventional 5% topical minoxidil treatment.

In 2020, Kochar et al. recognized that “tretinoin (TRET) is known for its comedolytic activity and is reported to enhance percutaneous absorption of minoxidil,” but both tretinoin and minoxidil “are being utilized for treatment of androgenic alopecia (AGA) in solution form which poses several problems in terms of poor solubility of drug, frequency of application and side effects.” Kochar et al. investigated “liposomal hydrogel system for simultaneous delivery of MXD and TRET to overcome the limitations of the existing formulation,” and reported “[a] ssociation with hydrogel enhanced the permeation of MXD through skin ex vivo but TRET [was] retained on the skin.” Kochar P, Nayak K, Thakkar S, et al. Exploring the potential of minoxidil tretinoin liposomal based hydrogel for topical delivery in the treatment of androgenic alopecia. Cutan Ocul Toxicol. 2020; 39 (1): 43-53.

Accordingly, in view of clinical trial in vivo data, it is surprising that the synergistic compositions disclosed herein comprising a retinoid agent in combination with minoxidil (and in some embodiments, a steroid) demonstrate enhanced hair growth.

### Steroid

In some embodiments, the composition further comprising a steroid in an amount ranging from about 0.01% to about 0.1% w/w, including about 0.01%, 0.02%, 0.03%, 0.04%, 0.05%, 0.06%, 0.07%, 0.08%, 0.09%, and 0.1% w/w steroid. In some embodiments, the composition comprises a steroid in an amount of 0.01% w/w, 0.015% w/w, 0.02% w/w, 0.025% w/w, 0.03% w/w, 0.035% w/w, 0.04% w/w, 0.045% w/w, 0.05% w/w, 0.055% w/w, 0.06% w/w, 0.065% w/w, 0.07% w/w, 0.075% w/w, 0.08% w/w, 0.085% w/w, 0.09% w/w, 0.095% w/w, or 0.10% w/w. The amount of steroid in the composition can help offset skin irritation of the combination of minoxidil and retinoic acid.

In some embodiments, the steroid comprises fluocinolone, triamcinolone, amcinonide, alclometasone dipropionate, betamethasone dipropionate, betamethasone valerate, clocortolone pivalate, clobetasone, clobetasol propionate, desoximetasone, diflucortolone valerate, desonide, halobetasol, diflorasone, diflorasone diacetate, propionate, flurandrenolide, fluocinonide, fluocinolone acetonide, halcinonide, hydrocortisone acetate, hydrocortisone hydrocortisone butyrate, hydrocortisone probutate, mometasone furoate, mapracorat, hydrocortisone acetate, methylprednisolone, prednicarbate, prednisolone, pefcalcitol, triamcinolone acetonide, or combinations thereof.

It is surprising that the synergistic compositions disclosed herein comprising a retinoid agent, minoxidil, and a steroid demonstrate enhanced hair growth. The combination with the steroid also increases likelihood of patient compliance with regular and long-term use.

Formulation

The compositions described herein are made via drug compounding, which is the process of combining and mixing two or more drugs.

The compositions of the present invention may be prepared in accordance with methods well known to the person skilled in the field of pharmacy.

For example, a topical solution, oil, or ointment formulation of the present disclosure can be prepared by methods known in the art, for example, standard compounding methods.

In some embodiments, the composition is formulated as a solution, spray, lotion, cream, gel, ointment, or as an emulsion, oil in water emulsion, or water in oil emulsion.

Compositions formulated as an ointment or an oil are preferred formulations for wavy, curly, or curly/kinky hair types. A composition formulated as an ointment spreads easily along frontal hairline and helps retain moisture. A composition formulated as an oil is ideal for thinning in the central scalp as it spreads easily and does not dry out hair.

In some embodiments, the composition comprises: minoxidil in an amount of 5%, adapalene in an amount of 0.1% w/w, and hydrocortisone in an amount of 1% w/w, wherein the composition is formulated as a solution, foam, or oil. Three to five drops should be applied once daily to thinning areas for women and up to twice daily for men.

In some embodiments, the composition comprises: minoxidil in an amount of 5%, retinol in an amount of 0.3% w/w, and hydrocortisone in an amount of 1% w/w, wherein the composition is formulated as a solution, foam, or oil. Three to five drops should be applied once daily to thinning areas for women and up to twice daily for men.

In some embodiments, the composition comprises: minoxidil in an amount ranging from 7% to 10% w/w, a retinoic acid in an amount of 0.025% w/w, and triamcinolone in an amount of 0.1% w/w.

In some embodiments, the composition comprises minoxidil in an amount ranging from 7% to 10% w/w, a retinoic acid in an amount of 0.025% w/w, and fluocinolone in an amount of 0.01% w/w.

In some embodiments, the composition comprises: minoxidil in an amount ranging from 7% to 10% w/w, a retinoic acid in an amount of 0.025% w/w, and fluocinolone in an amount of 0.05% w/w.

In some embodiments, the composition comprises minoxidil in an amount of 7% w/w, a retinoic acid in an amount of 0.025% w/w, and fluocinolone in an amount of 0.05% w/w, wherein the composition is formulated as a solution. This embodiment is preferred for those with straight hair as it evaporates very easily and it also minimizes the risk of irritation. Three to five drops should be applied once daily to thinning areas for women and up to twice daily for men.

In some embodiments, the composition comprises minoxidil in an amount of 7% w/w, a retinoic acid in an amount of 0.025% w/w, and fluocinolone in an amount of 0.01% w/w, wherein the composition is formulated as a solution. This embodiment is preferred for those with wavy hair because it is not too drying for this curl pattern and it has a residual moisturizing feel. Three to five drops should be applied once daily to thinning areas for women and up to twice daily for men.

In some embodiments, the composition comprises minoxidil in an amount of 7% w/w, a retinoic acid in an amount of 0.025% w/w, and fluocinolone in an amount of 0.01% w/w, wherein the composition is formulated as an oil. The oil base makes it easy to apply the composition to large, affected areas of the scalp. Three to five drops should be applied once daily to thinning areas for women and up to twice daily for men.

Minoxidil precipitates into a solid at concentrations higher than 7% w/w; and thus compositions comprising minoxidil in an amount higher than 7% w/w are better suited as cream, lotion, and ointment formulations.

In some embodiments, the composition comprises minoxidil in an amount of 8-10% w/w, a retinoic acid in an amount of 0.025% w/w, and triamcinolone in an amount of 0.1% w/w, wherein the composition is formulated as an ointment. In some embodiments, the composition comprises minoxidil in an amount of 10% w/w, retinoic acid in an amount of 0.025% w/w, and triamcinolone in an amount of 0.1% w/w, wherein the composition is formulated as an ointment. In some embodiments, the composition comprises minoxidil in an amount of 8% w/w, retinoic acid in an amount of 0.025% w/w, and clobetasol in an amount of 0.05% w/w, wherein the composition is formulated as an ointment or a cream. These embodiments are preferred for those with curly hair, especially those who have thinning along the frontal hairline. A pea sized amount should be applied once daily to affected areas.

Method of Treating Hair Loss

The presently disclosed subject matter provides a method of treating a hair loss condition, the method comprising applying a composition described herein to a subject's scalp.

In some embodiments, the subject has straight hair. In some embodiments, the subject has straight, wavy hair, curly hair, or curly/kinky hair. Hair type and curl pattern describe the specific shape of hair strands, and the way it naturally grows. Hair type is dependent on the shape of the hair follicle, the more oval shaped the hair follicle is, the curlier the hair will be. “Hair types,” or the classification of each hair's texture, is most often broken down into four main groups (1, 2, 3, and 4) describing curl shape, with “1” being Straight, “2” as Wavy, “3” Curly, and “4” as the Curly/Kinky. Further, each group further divided into three subcategories (a, b, and c) describing the width or diameter of the wave, curls, or coils with “a” being widest, “b” as medium, and “c” as the tightest curl. For example, 2C type hair is wavy, thick, and has S-bend curls that are well-defined and start at the roots; 3A type hair has large loose curls; 3B type hair has springy ringlets; and type 3C hair has tight corkscrew curls with strands densely packed together; 4A type hair has dense springy, S-patterned coils; 4B type hair has densely packed strands and can bend in sharp Z-angles; 4C type hair has tightly coiled strands with a tight zig-zag pattern, are more fragile, and have the most shrinkage. Type 4 curly/kinky hair is also referred to as Afro-textured or coily hair and is naturally very dry and spongy in texture, and can vary as soft, fine, coarse, or wiry. Type 4 hair strands form very tight small curls at the scalp and are prone to shrinkage. In some embodiments, the subject has Type 2C, 3A, 3B, 3C, 4A, 4B, or 4C hair.

In some embodiments, the subject has alopecia. In some embodiments, the subject has traction alopecia, androgenetic alopecia, or alopecia areata.

In some embodiments, the composition is applied as a pea sized amount. In some embodiments, the composition is applied once or twice daily to the scalp areas affected by hair loss. In some embodiments, the composition as described herein is applied once per day for at least 1, 2, 3, 4, 5, 6, 7, 8, 9, 10, 11, or 12 months. In some embodiments, the composition as described herein is applied once per day for at least 1, 2, 3, 4, or 5 years.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration and are not to be construed as limiting in any manner to make compounds, vesicles, or compositions of the disclosure by other methods.

The presently disclosed subject matter describes synergistic compositions for treating hair loss and related methods for treating hair loss.

The synergistic composition of minoxidil and retinoic acid as described herein displays a synergistic effect when applied in combination; that is, the components are more effective in combination than when applied individually. The synergistic compositions described enhance hair growth.

Patients received treatment with compositions comprising minoxidil, retinoic acid, and a topical steroid. Most of the patients had previously used OTC minoxidil 5% with suboptimal results.

### Example 1

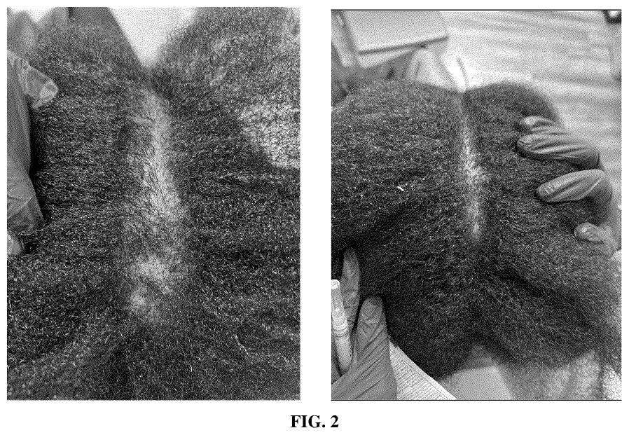

FIG. 2 are photos of a patient's central scalp (Patient 1) before treatment (left) and after treatment (right) with a composition comprising 7% minoxidil, 0.025% tretinoin and fluocinolone 0.01% oil. The composition was an oil. The patient applied a few drops to affected scalp areas once daily. The patient has type 4 curly/kinky hair and alopecia.

The composition comprising 7% minoxidil, 0.025% retinoic acid and fluocinolone 0.01% oil was prepared by compounding methods known in the art.

### Example 2

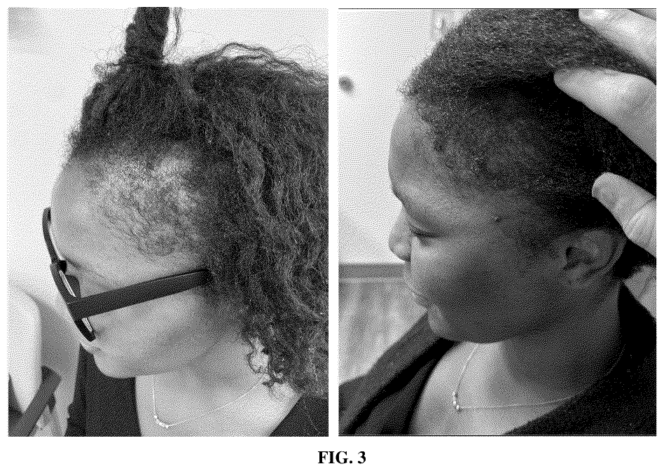

FIG. 3 are photos of a patient's scalp (Patient 2) before treatment (left) and after treatment (right) with a composition comprising 8% minoxidil, 0.025% tretinoin and triamcinolone 0.1% ointment for 4 months. The composition was an ointment. The patient applied a pea sized amount to affected scalp areas once daily. The patient has type 4 curly/kinky hair and alopecia. The compounded minoxidil in an ointment base enhances compliance over 4 months.

The composition comprising 8% minoxidil, 0.025% retinoic acid and triamcinolone 0.1% ointment was prepared by compounding methods known in the art.

### Example 3

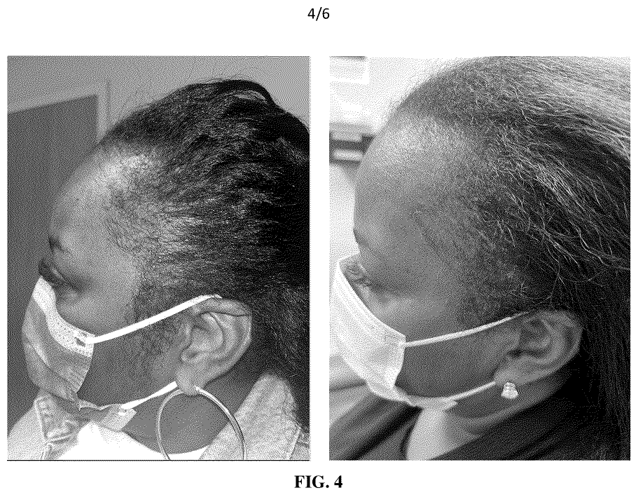

FIG. 4 are photos of a patient's scalp (Patient 3) before treatment (left) and after treatment (right) with a composition comprising 10% minoxidil, 0.025% tretinoin and triamcinolone 0.1% ointment for 4 months. The composition was an ointment. The patient applied a pea sized amount to the affected scalp areas once daily. The patient has type 4 curly/kinky hair and alopecia.

The composition comprising 10% minoxidil, 0.025% retinoic acid and triamcinolone 0.1% ointment was prepared by compounding methods known in the art.

### Example 4

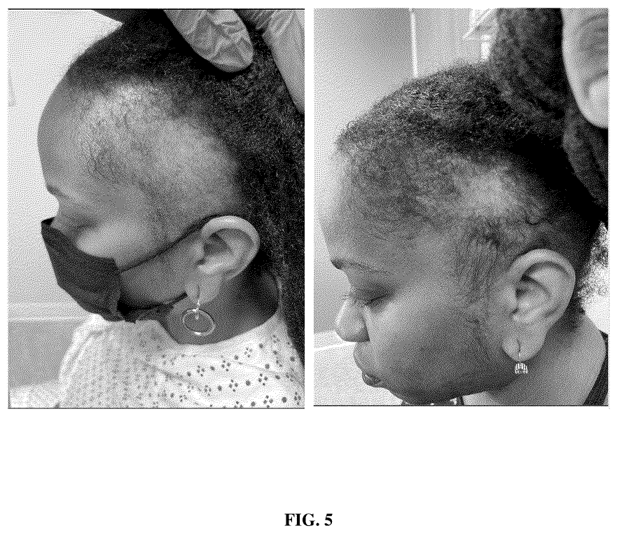

FIG. 5 are photos of a patient's scalp (Patient 4) before treatment (left) and after treatment (right) with a composition comprising 10% minoxidil, 0.025% tretinoin and triamcinolone 0.1% ointment. The composition was an ointment. The patient applied a pea sized amount to the affected scalp areas once daily. The patient has type 4 curly/kinky hair and alopecia.

The composition comprising 10% minoxidil, 0.025% retinoic acid and triamcinolone 0.1% ointment was prepared by compounding methods known in the art.

### Example 5

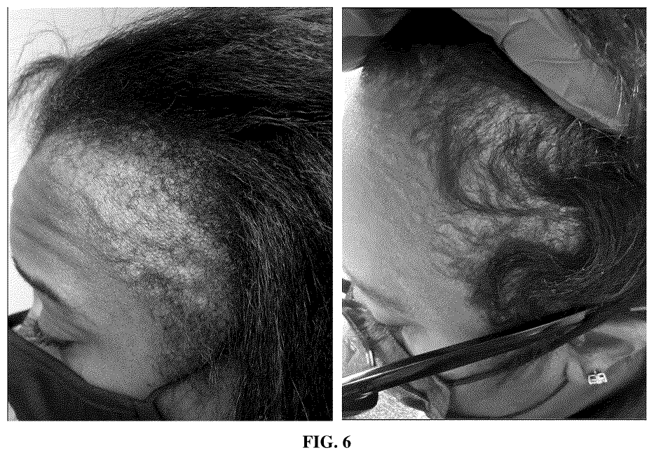

FIG. 6 are photos of a patient's scalp (Patient 5) before treatment (left) and after treatment (right) with a composition comprising 10% minoxidil, 0.025% tretinoin and triamcinolone 0.1% ointment for 1 year. The composition was an ointment. The patient applied a pea sized amount to affected scalp areas once daily. The patient has type 4 curly/kinky hair and alopecia.

The composition comprising 10% minoxidil, 0.025% retinoic acid and triamcinolone 0.1% ointment was prepared by compounding methods known in the art.

### Example 6

A comparative study is conducted with the following treatment types:

A: a composition comprising a composition comprising minoxidil in an amount of 8% w/w and a retinoic acid in an amount of 0.025% w/w;

B: a composition comprising a composition comprising minoxidil in an amount of 8% w/w, a retinoic acid in an amount of 0.025% w/w, and fluocinolone in an amount of 0.01% w/w;

C: a composition comprising a composition comprising minoxidil in an amount of 8% w/w, a retinoic acid in an amount of 0.025% w/w, and triamcinolone in an amount of 0.1% w/w; D: a composition comprising minoxidil in an amount of 5% w/w;

E: a composition comprising minoxidil in an amount of 5% w/w and tretinoin in an amount of 0.01% w/w;

F: a composition comprising minoxidil in an amount of 8% w/w and tretinoin in an amount of 0.01% w/w;

G: a composition comprising minoxidil in an amount of 8% w/w as the only active ingredient.

H: a composition comprising a retinoic acid in an amount of 0.025% w/w as the only active ingredient.

I: a composition comprising fluocinolone in an amount of 0.01% w/w as the only active ingredient.

J: a composition comprising triamcinolone in an amount of 0.1% w/w as the only active ingredient.

### REFERENCES

All publications, patent applications, patents, and other references mentioned in the specification and listed below are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

A.K. Gupta, M. Talukder, M. Venkataraman & M. A. Bamimore (2022) Minoxidil: a comprehensive review, Journal of Dermatological Treatment, 33:4, 1896-1906. Gupta et al. also discloses that efficacy of the minoxidil solution may not correlate with increased concentration.

Ghonemy S, Alarawi A, Bessar H. Efficacy and safety of a new 10% topical minoxidil versus 5% topical minoxidil and placebo in the treatment of male androgenetic alopecia: a trichoscopic evaluation. J Dermatolog Treat. 2021; 32 (2): 236-241.

Motamedi M, Chehade A, Sanghera R, Grewal P. A Clinician's Guide to Topical Retinoids. Journal of Cutaneous Medicine and Surgery. 2022;26 (1): 71-78.

Bazzano GS, Terezakis N, Galen W. Topical tretinoin for hair growth promotion. J Am Acad Dermatol. 1986; 15 (4 Pt 2): 880-893.

Ferry JJ, Forbes KK, VanderLugt JT, Szpunar GJ. Influence of tretinoin on the percutaneous absorption of minoxidil from an aqueous topical solution. Clin Pharmacol Ther. 1990 April;47 (4): 439-46.

Shin HS, Won CH, Lee SH, Kwon OS, Kim KH, Eun HC. Efficacy of 5% minoxidil versus combined 5% minoxidil and 0.01% tretinoin for male pattern hair loss: a randomized, double-blind, comparative clinical trial. Am J Clin Dermatol. 2007; 8 (5): 285-90.

Sharma A, Goren A, Dhurat R, et al. Tretinoin enhances minoxidil response in androgenetic alopecia patients by upregulating follicular sulfotransferase enzymes. Dermatol Ther. 2019; 32 (3): e12915

Kwon, O., S., Pyo, H., K., Oh, Y., J., Han, J., H., Lee, S., R., Chung, J., H., Eun, H., C., Kim, K., H. (April 2007). Promotive effect of minoxidil combined with all-trans retinoic acid (tretinoin) on human hair growth in vitro. Journal of Korean Medical Science, 22, 283-289.

Yoo, H., G., Chang, I., Y., Pyo, H., K., Kang, Y., J., Lee, S., H., Kwon, O., S., Cho, K., H., Eun, H., C., Kim, K., H. (January 2007). The additive effects of minoxidil and retinol on human hair growth in vitro. Biological and Pharmaceutical Bulletin, 30, 21-26.

Voorhees, J., J. (1990). Clinical effects of long-term therapy with topical tretinoin and cellular mode of action. Journal of International Medical Research, 18, 26-28.; Maiti, S., Chen, X., Chen, G. (2005). All-trans retinoic acid induction of sulfotransferases. Basic & Clinical Pharmacology & Toxicology, 96, 44-53.

Kochar P, Nayak K, Thakkar S, et al. Exploring the potential of minoxidil tretinoin liposomal based hydrogel for topical delivery in the treatment of androgenic alopecia. Cutan Ocul Toxicol. 2020; 39 (1): 43-53.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A synergistic composition for enhancing hair growth comprising:
minoxidil in an amount ranging from about 7% to about 10% w/w; and
a retinoid agent in an amount ranging from about 0.025% to about 0.1% w/w.

2. The composition of claim 1, wherein the retinoid agent comprises a retinoic acid selected from tazarotene, isotretinoin, adapalene, tretinoin, trifarotene, alitretinoin, and bexarotene.

3. The composition of claim 1, wherein the retinoid agent comprises retinol.

4. The composition of claim 1, further comprising a steroid in an amount ranging from about 0.01% to about 0.1% w/w.

5. The composition of claim 4, wherein the steroid is selected from fluocinolone, triamcinolone, amcinonide, alclometasone dipropionate, betamethasone dipropionate, betamethasone valerate, clocortolone pivalate, clobetasone, clobetasol propionate, desoximetasone, diflucortolone valerate, desonide, halobetasol, diflorasone, diflorasone diacetate, propionate, flurandrenolide, fluocinonide, fluocinolone acetonide, halcinonide, hydrocortisone acetate, hydrocortisone valerate, hydrocortisone butyrate, hydrocortisone probutate, mometasone furoate, mapracorat, hydrocortisone acetate, methylprednisolone, prednicarbate, prednisolone, pefcalcitol, triamcinolone acetonide, and combinations thereof.

6. The composition of claim 1, wherein the composition is formulated as a solution, spray, lotion, cream, gel, ointment, or as an emulsion, oil in water emulsion, or water in oil emulsion.

7. The composition of claim 1, wherein the composition comprises:
minoxidil in an amount ranging from about 7% to about 10% w/w;
a retinoic acid in an amount of about 0.025% w/w; and
triamcinolone in an amount of about 0.1% w/w.

8. The composition of claim 1, wherein the composition comprises:
minoxidil in an amount ranging from about 7% to about 10% w/w;
a retinoic acid in an amount of about 0.025% w/wl and fluocinolone in an amount of about 0.01% w/w.

9. The composition of claim 1, wherein the composition comprises:
minoxidil in an amount ranging from about 7% to about 10% w/w;
a retinoic acid in an amount of about 0.025% w/w; and
fluocinolone in an amount of about 0.05% w/w.

10. The composition of claim 1, wherein the composition comprises:
minoxidil in an amount of about 7% w/w;
a retinoic acid in an amount of about 0.025% w/w; and
fluocinolone in an amount of about 0.01% w/w;
wherein the composition is formulated as an oil.

11. The composition of claim 1, wherein the composition comprises:
minoxidil in an amount of about 8% w/w;
a retinoic acid in an amount of about 0.025% w/w; and
triamcinolone in an amount of about 0.1% w/w;
wherein the composition is formulated as an ointment.

12. The composition of claim 1, wherein the composition comprises:
minoxidil in an amount of about 10% w/w;
retinoic acid in an amount of about 0.025% w/w; and
triamcinolone in an amount of about 0.1% w/w;
wherein the composition is formulated as an ointment.

13. A method for treating a hair loss condition, the method comprising:
applying the composition of claim 1 to a subject's scalp.

14. The method of claim 13, wherein the subject has straight hair, wavy hair, or curly hair.

15. The method of claim 13, wherein the subject has alopecia.

16. The method of claim 13, wherein the composition of claim 1 is applied once per day.

17. The method of claim 13, wherein the composition of claim 1 is applied once per day for at least 4 months.

18. The method of claim 13, wherein the composition of claim 1 is applied once per day for at least 1 year.
